(19) United States
(12) Patent Application Publication
(10) Pub. No.: US 2003/0198633 A1
(43) Pub. Date: Oct. 23, 2003

(54) Methods for sustained drug delivery and compositions useful therefor

(75) Inventors: Robert E. Jordan, Malvern, PA (US); David M. Knight, Berwyn, PA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Centocor, Inc., Malvern, PA

(21) Appl. No.: 10/406,083

(22) Filed: Apr. 2, 2003

Related U.S. Application Data
(63) Continuation of application No. 08/801,411, filed on Feb. 19, 1997, now abandoned.

(51) Int. Cl. A61K 39/395; A61K 31/727; C07K 16/46

(52) U.S. Cl. 424/130.1; 514/56; 530/391.1

(57) ABSTRACT

Methods for sustained delivery of a therapeutic agent to the circulation of a patient are disclosed. Also disclosed are methods of preparing bioconjugates for sustained delivery of a therapeutic agent to the circulation of a patient.

### RELATED APPLICATION

This application is a continuation of U.S. application Ser. No. 08/801,411, filed Feb. 19, 1997. The entire teachings of the above application are incorporated herein by reference.

### BACKGROUND OF THE INVENTION

Many therapeutic agents have undesirably short pharmacokinetic lifetimes. As a result, they must be administered in large amounts or administered continuously or on a repeated basis to maintain the desired effect.

### SUMMARY OF THE INVENTION

The invention provides methods for sustained delivery of a therapeutic agent to the circulation of a patient. In one embodiment, the method comprises administering to the patient a predetermined effective amount of the bioconjugate, the bioconjugate comprising a binding moiety and a therapeutic agent. In a second embodiment, the method comprises (a) administering to the patient a predetermined effective amount of a first bioconjugate comprising a binding moiety and a capture moiety, said capture moiety comprising a binding site for a complementary binding partner; and (b) administering to the patient a predetermined effective amount of a second bioconjugate comprising a complementary binding partner and the therapeutic agent.

The invention further relates to methods of preparing a bioconjugate for sustained delivery of a therapeutic agent to the circulation of a patient. In one embodiment, the method of preparing a bioconjugate for sustained delivery of a therapeutic agent to the circulation of a patient comprises (a) conjugating a binding moiety to a therapeutic agent, thereby producing a bioconjugate; and (b) screening said bioconjugate for sustained delivery of the therapeutic agent. In a second embodiment, the method comprises (a) selecting a binding moiety; (b) selecting a therapeutic agent; (c) conjugating the binding moiety to the therapeutic agent, thereby producing a bioconjugate; and (d) screening said bioconjugate for sustained delivery of the therapeutic agent. In a third embodiment, the method of preparing a bioconjugate for sustained delivery of a therapeutic agent to the circulation of a patient comprises (a) conjugating a binding moiety to a capture moiety, thereby producing a bioconjugate; and (b) screening said bioconjugate for sustained delivery to the circulation of the patient.

The invention also relates to novel bioconjugates and their use for sustained delivery of a therapeutic agent to the circulation of a patient.

The invention further relates to bioconjugates and their use in the manufacture of medicaments for sustained delivery to the circulation of a patient.

Binding moieties useful in the invention include binding moieties that bind to a platelet, such as anti-platelet antibodies and antigen-binding fragments thereof. Binding moieties useful in the invention also include binding moieties that bind to a red cell, such as anti-red cell antibodies and antigen-binding fragments thereof.

In one embodiment of the invention, the binding moiety is an antibody or antibody fragment that binds to a glycoprotein IIb/IIIa receptor. In another embodiment of the invention, the binding moiety is an antibody or antibody fragment which competitively inhibits the binding of a murine 7E3 antibody or an antigen-binding fragment thereof to a platelet. In a particular embodiment of the invention, the binding moiety is a chimeric 7E3 antibody or an antigen-binding fragment thereof. In a preferred embodiment of the invention, the binding moiety is a chimeric 7E3 Fab fragment (also referred to as abciximab or ReoPro® antibody) or a chimeric 7E3 Fab′ fragment. Chimeric 7E3 Fab is presently available from Centocor, Inc. (Malvern, Pa.) and/or Eli Lilly & Co. (Indianapolis, Ind.).

Therapeutic agents useful in the invention are those agents which can provide a patient with a therapeutic advantage from reduced dose or prolonged circulation in the patient which can be achieved according to the present invention. Such therapeutic agents include small molecules, proteins, antibodies and antigen-binding fragments thereof. In a particular embodiment, the therapeutic agent is heparin.

Capture moieties useful in the invention are members of a specific binding pair and comprise a binding site for a complementary binding partner. Such capture moieties include antibodies/antigens, hormones/receptors, and other binding pairs (e.g., avidin/biotin).

### BRIEF DESCRIPTION OF THE DRAWINGS

FIGS. 1A and 1B are a series of fluorescence histograms showing the distribution of platelet-bound abciximab in a patient who received a 0.25 mg/kg bolus plus a 0.125 μg/kg/min infusion for 12 hours (FIG. 1A) and in a patient who received 0.25 mg/kg bolus plus a 10 μg/minute infusion for 12 hours (FIG. 1B), as measured by flow cytometric assay.

FIG. 2 is a plot showing the persistence of abciximab on platelets as measured by the fluorescence values obtained from each patient at 8 and 15 days after abciximab administration.

FIG. 3A is a graph showing molecules of abciximab bound per platelet after treatment of platelets with varying concentrations of radiolabeled abciximab, as measured by radiometric assay.

FIG. 3B is a graph showing median fluorescence intensity of platelets after treatment with varying concentrations of abciximab, as measured by flow cytometric assay using FITC-labeled anti-abciximab to detect bound antibody.

FIG. 4 is a graph showing the final linear regression analysis correlating molecules of abciximab bound per platelet with observed level of fluorescence intensity.

FIG. 5 is a plot showing calculated values for receptor occupancy (molecules of abciximab bound per platelet) in each patient at 8 and 15 days after abciximab administration.

### DETAILED DESCRIPTION OF THE INVENTION

Chimeric 7E3 Fab fragment binds rapidly to platelets but dissociates slowly and then continually redistributes amoung circulating platelets. As a monovalent Fab fragment, each molecule of chimeric 7E3 Fab binds rapidly and with high affinity to a single glycoprotein IIb/IIIa receptor (K_{d}˜5 nM). The dissociation rate of chimeric 7E3 Fab from the platelet surface is slow and occurs over several hours in vitro. In blood samples obtained from treated patients, chimeric 7E3 Fab can be detected on platelet surfaces for longer (≧2 weeks) than the circulating lifespan of platelets (˜7-9 days). The surprising prolonged circulation of platelet-bound chimeric 7E3 Fab is believed to be due to a continuous redistribution among all circulating platelets resulting in a uniformly-coated population of platelets with gradually decreasing levels of glycoprotein IIb/IIIa receptor blockade. At 15 days following treatment, approximately 10,000 molecules of chimeric 7E3 Fab typically are detected on the surface of each circulating platelet. Thus, the pharmacodynamic profile of chimeric 7E3 results in persistent binding to platelets and a gradual and tapered recovery from early profound levels of receptor blockade.

The rate of dissociation is an inherent property of the monovalent 7E3 Fab fragment and is not shared by the bivalent 7E3 F(ab′)₂ fragment which dissociates at a nearly undetectable rate. The slow dissociation rate is a function of the basic thermodynamic binding parameters of the 7E3 combining site with the glycoprotein IIb/IIIa receptor. After injection into a patient, chimeric 7E3 Fab dissociates over time and a fraction of this dissociated antibody continually redistributes among circulating platelets. About 10-15% of all circulating platelets are newly synthesized and secreted every 24 hours and redistribution of chimeric 7E3 Fab onto “new” platelets is continually occurring. As a result of continuous redistribution, chimeric 7E3 Fab persists on circulating platelets beyond the average lifespan of the platelet.

Additional binding moieties can be screened for pharmacodynamic behavior similar to that described herein for chimeric 7E3 Fab. Chimeric 7E3 Fab, Fab′, or other suitable binding moieties can also be incorporated into a bioconjugate and the resulting bioconjugate screened for pharmacodynamic behavior similar to that described herein for chimeric 7E3 Fab.

Advantageously, conjugates of chimeric 7E3 Fab (or other binding moiety) and an agent, such as a therapeutic agent, coupled to it will share the gradual, tapered pharmacodynamic disappearance of platelet-bound c7E3 Fab from circulation. Such conjugates of chimeric 7E3 Fab (or other binding moiety) and an agent coupled to it will have a prolonged circulating lifetime since clearance of the agent from the circulation will be delayed due to its conjugation to chimeric 7E3 Fab (or other binding moiety) which binds platelets (or other suspended formed elements of the blood) with high affinity. Such conjugates will enable sustained delivery of a therapeutic agent to the circulation of a patient. In a particular embodiment, sustained presence in the circulation (prolonged circulating lifetime) for up to about two weeks, and preferably about three weeks, following a single injection can be achieved. The term “sustained delivery” of a therapeutic agent to the circulation of a patient refers to prolonged circulation of the therapeutic agent in the patient.

The benefits of prolonging circulating lifetime (or delaying clearance from circulation) of therapeutic agents include high clinical response rates for significantly longer durations in comparison with that obtained with treatment with therapeutic agents with shorter circulating lifetimes. In addition, lower dosages can be administered to provide the same therapeutic response, thus increasing the therapeutic window between a therapeutic and a toxic effect. Lower doses may also result in lower financial costs to the patient, and potentially fewer side effects. Fewer side effects further enable administration of multiple dosages of agent with enhanced safety.

The present invention relates to methods for sustained delivery of a therapeutic agent to the circulation of a patient. In one embodiment, the method for sustained delivery of a therapeutic agent to the circulation of a patient comprises administering to the patient a predetermined effective amount of a bioconjugate, the bioconjugate comprising a binding moiety and a therapeutic agent.

In a second embodiment, the method for sustained delivery of a therapeutic agent to the circulation of a patient comprises (a) administering to the patient a predetermined effective amount of a first bioconjugate comprising a binding moiety and a capture moiety, said capture moiety comprising a binding site for a complementary binding partner; and (b) administering to the patient a predetermined effective amount of a second bioconjugate comprising a complementary binding partner and the therapeutic agent.

The invention also relates to methods of preparing a bioconjugate for sustained delivery of a therapeutic agent to the circulation of a patient. In one embodiment, the method of preparing a bioconjugate for sustained delivery of a therapeutic agent to the circulation of a patient comprises (a) conjugating a binding moiety to a therapeutic agent, thereby producing a bioconjugate; and (b) screening said bioconjugate for sustained delivery of the therapeutic agent. In a second embodiment, the method comprises (a) selecting a binding moiety; (b) selecting a therapeutic agent; (c) conjugating the binding moiety to the therapeutic agent, thereby producing a bioconjugate; and (d) screening said bioconjugate for sustained delivery of the therapeutic agent. In a third embodiment, the method of preparing a bioconjugate for sustained delivery of a therapeutic agent to the circulation of a patient comprises (a) conjugating a binding moiety to a capture moiety, thereby producing a bioconjugate; and (b) screening said bioconjugate for sustained delivery to the circulation of the patient.

The term circulation is meant to refer to blood circulation. The term blood refers to the “circulating tissue” of the body, the fluid and its suspended formed elements that are circulated through the heart, arteries, capillaries and veins. The suspended formed elements of the blood include red blood cells (red cells, erythrocytes), white blood cells (leukocytes) and platelets.

Binding Moieties

The term “binding moiety”, as used herein, refers to an agent which selectively binds to suspended formed elements of the blood. A binding moiety which selectively binds to a red cell can be advantageous because of the approximately 4 month lifetime of the red cell. A binding moiety which selectively binds to a leukocyte can be advantageous because of the unique cellular functions of the leukocyte. In a preferred embodiment, the binding moiety has a pharmacodynamic profile similar to that described herein for chimeric 7E3 Fab (persistent binding to a particular class of suspended formed elements, slow dissociation from the surface of the suspended formed element, continuous redistribution among circulating suspended formed elements of the class). For example, the binding moiety can be an antibody, an antigen-binding antibody fragment, a peptide or a ligand of a surface receptor.

For example, the binding moiety can be an antibody which selectively binds the desired antigen, such as a platelet surface antigen such as glycoprotein IIb/IIIa. In a preferred embodiment, the antibodies specifically bind the antigen. The antibodies can be polyclonal or monoclonal, and the term antibody is intended to encompass both polyclonal and monoclonal antibodies. The terms polyclonal and monoclonal refer to the degree of homogeneity of an antibody preparation, and are not intended to be limited to particular methods of production.

Suitable antibodies are available, or can be raised against an appropriate immunogen, such as isolated and/or recombinant antigen or portion thereof (including synthetic molecules, such as synthetic peptides) or against a host cell which expresses recombinant antigen. In addition, cells expressing recombinant antigen, such as transfected cells, can be used as immunogens or in a screen for antibody which binds receptor (see e.g., Chuntharapai et al., J. Immunol., 152: 1783-1789 (1994); Chuntharapai et al., U.S. Pat. No. 5,440,021).

Preparation of immunizing antigen, and polyclonal and monoclonal antibody production can be performed using any suitable technique. A variety of methods have been described (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel et al., Eds., (John Wiley & Sons: New York, N.Y.), Chapter 11, (1991)). Generally, a hybridoma can be produced by fusing a suitable immortal cell line (e.g., a myeloma cell line such as SP2/0) with antibody producing cells. The antibody producing cell, preferably those of the spleen or lymph nodes, can be obtained from animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

Other suitable methods of producing or isolating antibodies of the requisite specificity, including human antibodies, can be used, including, for example, methods by which a recombinant antibody or portion thereof are selected from a library (e.g., Hoogenboom et al., WO 93/06213; Hoogenboom et al:, U.S. Pat. No. 5,565,332; WO 94/13804, published Jun. 23, 1994; Krebber et al., U.S. Pat. No. 5,514,548; and Dower et al., U.S. Pat. No. 5,427,908), or which rely upon immunization of transgenic animals (e.g., mice) capable of producing a full repertoire of human antibodies (see e.g., Jakobovits et al., Proc. Natl. Acad. Sci. USA, 90: 2551-2555 (1993); Jakobovits et al., Nature, 362: 255-258 (1993); Kucherlapati et al., European Patent No. EP 0 463 151 B1; Lonberg et al., U.S. Pat. No. 5,569,825; Lonberg et al., U.S. Pat. No. 5,545,806; and Surani et al., U.S. Pat. No. 5,545,807).

Single chain antibodies, and chimeric, humanized or primatized (CDR-grafted antibodies, with or without framework changes), or veneered antibodies, as well as chimeric, CDR-grafted or veneered single chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., U.S. Pat. No. 5,585,089; Queen et al., European Patent No. 0,451,216 B1; Adair et al., WO 91/09967, published Jul. 11, 1991; Adair et al., European Patent No. 0,460,167 B1; and Padlan, E. A. et al., European Patent No. 0,519,596 A1. See also, Newman, R. et al., BioTechnology, 10: 1455-1460 (1992), regarding primatized antibody, and Huston et al., U.S. Pat. No. 5,091,513; Huston et al., U.S. Pat. No. 5,132,405; Ladner et al., U.S. Pat. No. 4,946,778 and Bird, R. E. et al., Science, 242: 423-426 (1988)) regarding single chain antibodies.

In addition, antigen binding fragments of antibodies, including fragments of chimeric, humanized, primatized, veneered or single chain antibodies and the like, can also be produced. For example, antigen binding fragments include, but are not limited to, fragments such as Fv, Fab, Fab′ and F(ab′)₂ fragments. Antigen binding fragments can be produced by enzymatic cleavage or by recombinant techniques, for example. For instance, papain or pepsin cleavage can generate Fab or F(ab′)₂ fragments, respectively. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain.

In a preferred embodiment, the binding moiety has binding specificity for a platelet surface antigen, such as glycoprotein IIb/IIIa, GMP-140, or another platelet surface antigen. For example, platelet binding agents, including GPIIb/IIIa antagonists, such as anti-GPIIb/IIIa antibodies (wherein the term “antibody” is as defined herein), peptide antagonists, such as snake venom proteins and their derivatives (e.g., disintegrins, integrelin), and non-peptide compounds or peptidomimetics, such as Ro 44-9883 (Hoffman-LaRoche), MK-383 (Merck), SC54684 (Searle), or other anti-platelet agents (see e.g., Coller, B. S. et al., “New Antiplatelet Agents: Platelet GPIIb/IIIa Antagonists,” Thrombosis and Haemostasis, 74 (1): 302-308 (1995); Cook, J. S. et al., “Platelet glycoprotein IIb/IIIa antagonists,” Drugs of Future, 19: 135-139 (1994); and Cox, D. et al., “The pharmacology of integrins”, Medicinal Research Reviews, 14: 195-228 (1994)), can be assessed for use in the present method.

Preferably, the binding moiety has binding specificity for glycoprotein IIb/IIIa (also referred to as GPIIb/IIIa or CD41/CD61), and even more preferably, the binding moiety is an antibody or antigen binding fragment thereof. Such antibodies or fragments can be obtained as described above. Antibodies reactive with glycoprotein IIb/IIIa can be raised against a suitable immunogen such as platelets, isolated and/or purified GPIIb/IIIa, or its component chains, especially the β₃ chain, portions of the foregoing or synthetic molecules, such as synthetic peptides.

In a particularly preferred embodiment, the antibody or antigen binding fragment thereof is murine or chimeric 7E3 (or an antigen binding fragment thereof), or has an epitopic specificity similar to that of murine or chimeric 7E3, or antigen binding fragments thereof, including antibodies or antigen binding fragments reactive with the same or a functionally equivalent epitope on GPIIb/IIIa as that bound by murine or chimeric 7E3, or antigen binding fragments thereof (see, EP 0,205,207; EP 0,206,532; EP 0,206,533 B1; Coller et al., U.S. Ser. No. 08/375,074, filed Jan. 17, 1995; and Coller et al., WO 95/12412, published May 11, 1995, the teachings of which are each incorporated herein by reference in their entirety). Murine hybridoma 7E3 was deposited on May 30, 1985 at the American Type Culture Collection, 10801 University Boulevard, Manassa, Va. 20110, and is available under accession number HB 8832. The 7E3 antibody has specificity for GPIIb/IIIa. The 7E3 antibody also cross-reacts with the vitronectin receptor (αᵥβ₃, also referred to as CD51/CD61), an integrin which uses the same β subunit (i.e., β₃) as GPIIb/IIIa but has a different a subunit. The vitronectin receptor is expressed on cells such as endothelial cells and vascular smooth muscle cells (and to a lesser extent, on platelets), and mediates adhesion to a variety of extracellular matrix proteins (e.g., vitronectin, fibronectin, von Willebrand Factor, fibrinogen, osteopontin, thrombospondin, collagen, perlecan). Antibodies with an epitopic specificity similar to that of c7E3 Fab or the 7E3 monoclonal antibody include antibodies which can compete with murine or chimeric 7E3 (or antigen binding fragments thereof) for binding to platelet GPIIb/IIIa (see e.g., Coller et al., U.S. Ser. No. 08/375,074, filed Jan. 17, 1995; Coller et al., WO 95/12412, published May 11, 1995). In a preferred embodiment, such a cross-reactive antibody or portion thereof (e.g., a Fab or Fab′ fragment) persists in the circulation, redistributing to circulating platelets.

In another embodiment, the binding moiety has binding specificity for a red cell surface antigen. For example, red cell binding agents, including anti-red cell antibodies and antigen-binding fragments thereof, peptide antagonists, and non-peptide compounds or peptidomimetics, or other anti-red cell agents, can be assessed for use in the present method.

Capture Moieties and Complementary Binding Partners

The term “capture moiety”, as used herein, refers to a member of a specific binding pair and comprises a binding site for a complementary binding partner. Suitable capture moieties and complementary binding partners can be obtained from specific binding pairs including antibody/antigen, hormone/receptor, and other binding pairs (e.g., avidin/biotin).

Therapeutic Agents

The term “therapeutic agent”, as used herein, refers to an agent which can provide a patient with a therapeutic advantage from reduced dose or prolonged circulation in the patient which can be achieved according to the present method. The therapeutic agent need not act at the site bound by the binding moiety and usually does not. Thus, the binding moiety is selected to achieve sustained delivery, rather than localization of the therapeutic agent to a particular site of action. Therapeutic benefit occurs as a result of prolonged circulation of the therapeutic agent in the patient and not as a result of action of the therapeutic agent at the site bound by the binding moiety.

In a particular embodiment, the therapeutic agent can bind to a target circulating in the circulation of the patient. Prolonged circulation of the therapeutic agent in the patient provides the patient with a therapeutic advantage.

In another embodiment, the therapeutic agent has a short pharmacokinetic lifetime. As discussed herein, to prolong the circulating lifetime of the therapeutic agent (delay clearance from circulation, lengthen pharmacokinetic lifetime), the therapeutic agent can be conjugated to a binding moiety with a pharmacodynamic profile similar to that described herein for chimeric 7E3 Fab. Advantageously, the resulting bioconjugate will have the prolonged pharmacodynamics of the binding moiety.

Therapeutic agents can be, for example, proteins, peptides, glycoproteins, lipoproteins, phospholipids, steroids, steroid analogs, alkaloids, vitamins, saccharides and genetic material, including nucleosides, nucleotides and polynucleotides. Therapeutic agents include antibodies and antigen-binding antibody fragments, enzymes, lymphokines, growth factors, immune modulators, thrombolytic agents, such as, but not limited to, tissue plasminogen activator, insulin, hormones, agents that enhance erythropoiesis, such as erythropoietin, anticoagulants and antithombotics, such as, but not limited to, heparin, antithrombin, hirudin, anti-tissue factor agents and anti-Factor VII agents, anti-proliferative agents, anti-cytokines, such as, but not limited to, tumor necrosis factor antagonists, such as, but not limited to, anti-tumor necrosis factor antibodies, receptor molecules which bind specifically to tumor necrosis factor and other anti-tumor necrosis factor agents, stimulatory cytokines, anti-immune cell receptor targets, such as, but not limited to, CD4 receptor targets, agents that stimulate or oppose wound healing, procoagulants, including those suitable for hemophilia therapy, such as, but not limited to, Factor VIII and Factor IX, proteinase inhibitors, such as, but not limited to, metalloproteinase inhibitors and alpha-1 proteinase inhibitors, and anti-cancer agents.

In a particular embodiment, the therapeutic agent is the anticoagulant heparin. Presently available formulations of heparin include TUBEX® heparin lock flush solution, USP, heparin flush kit and TUBEX® heparin sodium injection, USP (Wyeth-Ayerst Laboratories, Philadelphia, Pa.); heparin sodium injection, USP (Eli Lilly & Co., Indianapolis, Ind.); and heparin sodium injection, USP, HEP-LOCK® (heparin lock flush solution, USP) and HEP-LOCK® dorsette cartridge needle units (Elkins-Sinn, Inc., Cherry Hill, N.J.).

In another embodiment, the therapeutic agent is an anti-tumor necrosis factor antibody or antigen-binding fragment thereof. Antibodies or antigen binding fragments are as described above. As used herein, an “anti-tumor necrosis factor antibody” decreases, blocks, inhibits, abrogates or interferes with tumor necrosis factor (TNF) activity in vivo. In a particular embodiment, the antibody or antigen binding fragment thereof is chimeric monoclonal antibody cA2 (or an antigen binding fragment thereof), or has an epitopic specificity similar to that of chimeric antibody cA2, murine monoclonal antibody A2, or antigen binding fragments thereof, including antibodies or antigen binding fragments reactive with the same or a functionally equivalent epitope on human TNFα as that bound by chimeric antibody cA2 or murine monoclonal antibody A2, or antigen binding fragments thereof. Antibodies with an epitopic specificity similar to that of chimeric antibody cA2 or murine monoclonal antibody A2 include antibodies which can compete with chimeric antibody cA2 or murine monoclonal antibody A2 (or antigen binding fragments thereof) for binding to human TNFα. Such antibodies or fragments can be obtained as described above. Chimeric antibody cA2, murine monoclonal antibody A2 and methods of obtaining these antibodies are also described in U.S. application Ser. No. 08/192,093 (filed Feb. 4, 1994; now U.S. Pat. No. 6,284,471), U.S. application Ser. No. 08/192,102 (filed Feb. 4, 1994; now U.S. Pat. No. 5,656,272), U.S. application Ser. No. 08/192,861 (filed Feb.4, 1994; now U.S. Pat. No. 5,919,452), U.S. application Ser. No. 08/324,799 (filed Oct. 18, 1994; now U.S. Pat. No. 5,698,195), Le, J. et al., International Publication No. WO 92/16553 (published Oct. 1, 1992), Knight, D. M. et al., Mol. Immunol. 30:1443-1453 (1993), and Siegel, S. A. et al., Cytokine 7(1):15-25 (1995), which references are each entirely incorporated herein by reference.

Chimeric antibody cA2 consists of the antigen binding variable region of the high-affinity neutralizing mouse anti-human TNF IgG1 antibody, designated A2, and the constant regions of a human IgG1, kappa immunoglobulin. The human IgG1 Fc region improves allogeneic antibody effector function, increases the circulating serum half-life and decreases the immunogenicity of the antibody. The avidity and epitope specificity of the chimeric antibody cA2 is derived from the variable region of the murine antibody A2. In a particular embodiment, a preferred source for nucleic acids encoding the variable region of the murine antibody A2 is the A2 hybridoma cell line.

Chimeric A2 neutralizes the cytotoxic effect of both natural and recombinant human TNF in a dose dependent manner. From binding assays of chimeric antibody cA2 and recombinant human TNF, the affinity constant of chimeric antibody cA2 was calculated to be 1.8×10⁹M⁻¹. Preferred methods for determining mAb specificity and affinity by competitive inhibition can be found in Harlow, et al., Antibodies: A Laboratory Manual, Cold Spring Harbor Laboratory Press, Cold Spring Harbor, N.Y., 1988; Colligan et al., eds., Current Protocols in Immunology, Greene Publishing Assoc. and Wiley Interscience, New York, (1992, 1993); Kozbor et al., Immunol. Today 4:72-79 (1983); Ausubel et al., eds. Current Protocols in Molecular Biology, Wiley Interscience, New York (1987, 1992, 1993); and Muller, Meth. Enzymol. 92:589-601 (1983), which references are entirely incorporated herein by reference.

In a particular embodiment, murine monoclonal antibody A2 is produced by a cell line designated c134A. Chimeric antibody cA2 is produced by a cell line designated c168A.

Bioconjugate Isolation

The term bioconjugate is meant to refer to any complex comprising a binding moiety and a therapeutic agent or capture moiety and any complex comprising a complementary binding partner and a therapeutic agent, wherein the individual components of each bioconjugate are different from each other. In a preferred embodiment, bioconjugates useful in the present invention have a pharmacodynamic profile similar to that described herein for chimeric 7E3 Fab (persistent binding to a particular class of suspended formed elements, slow dissociation from the surface of the suspended formed element, continuous redistribution among circulating suspended formed elements of the class). A variety of methods for preparing and isolating (e.g., purifying) bioconjugates have been described (see, e.g., Hermanson, G. T., Bioconjugate Techniques, Academic Press, San Diego, Calif. (1996); Bode et al., EP 0 465 556 B1, published Jan. 15, 1992; Bode et al., WO 90/11783, published Oct. 18, 1990; Chang et al., WO 90/06133, published Jun. 14, 1990; Neblock et al., Bioconjugate Chem., 3: 126-131 (1992); Wagner et al., Blood, 88(3): 907-914 (1996); Griffiths et al., WO 96/40245, published Dec. 19, 1996; Haber et al., U.S. Pat. No. 5,453,269; and Haber et al., U.S. Pat. No. 5,443,827, which references are entirely incorporated herein by reference). These or other suitable methods can be used to prepare a desired bioconjugate.

A bioconjugate has the combined properties of its individual components, which are conjugated (linked) together. The linkage can be noncovalent or covalent and can be direct or indirect (e.g., via a linker). The individual components can be conjugated using chemical, cell fusion or recombinant techniques (see, e.g., Hermanson, G. T., Bioconjugate Techniques, Academic Press, San Diego, Calif. (1996); Bode et al., EP 0 465 556 B1, published Jan. 15, 1992; Bode et al., WO 90/11783, published Oct. 18, 1990; Chang et al., WO 90/06133, published Jun. 14, 1990; Neblock et al., Bioconjugate Chem., 3: 126-131 (1992); Wagner et al., Blood, 88(3): 907-914 (1996); Griffiths et al., WO 96/40245, published Dec. 19, 1996; Haber et al., U.S. Pat. No. 5,453,269; and Haber et al., U.S. Pat. No. 5,443,827, which references are entirely incorporated herein by reference).

For example, in a particular embodiment, the bioconjugate comprises a binding moiety that is an antibody or an antigen-binding antibody fragment and a therapeutic agent that is also an antibody or an antigen-binding antibody fragment. A bioconjugate comprising two antibody components is also referred to as an immunoconjugate and more particularly as a heterobifunctional or bispecific antibody. A heterobifunctional antibody can be isolated in a variety of ways (see, e.g., Chang et al., WO 90/06133 (published Jun. 14, 1990); Neblock et al., Bioconjugate Chem., 3: 126-131 (1992); Wagner et al., Blood, 88(3): 907-914 (1996); Haber et al., U.S. Pat. No. 5,453,269); and Haber et al., U.S. Pat. No. 5,443,827). The two antibody components can be linked using chemical, cell fusion or recombinant techniques. The linkage can be noncovalent but is preferably covalent. Chang et al., WO 90/06133 (published Jun. 14, 1990); Neblock et al., Bioconjugate Chem., 3: 126-131 (1992); Wagner et al., Blood, 88(3): 907-914 (1996); Haber et al., U.S. Pat. No. 5,453,269); and Haber et al., U.S. Pat. No. 5,443,827, which references are entirely incorporated herein by reference, provide several methods for conjugating antibody components.

In another embodiment, the bioconjugate comprises a binding moiety that is an antibody or an antigen-binding antibody fragment and a therapeutic agent that is not an antibody or an antigen-binding antibody fragment. A bioconjugate comprising at least one antibody component is also referred to as an immunoconjugate. An immunoconjugate can be isolated in a variety of ways (see, e.g., Bode et al., EP 0465 556 B1 (published Jan. 15, 1992); Bode et al., WO 90/11783 (published Oct. 18, 1990); and Hermanson, G. T., Bioconjugate Techniques, Academic Press, San Diego, Calif. (1996)). The antibody and non-antibody components can be linked using chemical or recombinant techniques. The linkage can be noncovalent but is preferably covalent. Bode et al., EP 0 465 556 B1 (published Jan. 15, 1992); Bode et al., WO 90/11783 (published Oct. 18, 1990); and Hermanson, G. T., Bioconjugate Techniques, Academic Press, San Diego, Calif. (1996), both of which are entirely incorporated herein by reference, provide several methods for conjugating antibody and non-antibody components.

In yet another embodiment, the bioconjugate comprises a binding moiety and a capture moiety or a therapeutic agent or comprises a complementary binding partner and a therapeutic agent. Such bioconjugates can be isolated using a variety of techniques (see, e.g., Griffiths et al., WO 96/40245 (published Dec. 19, 1996) and Hermanson, G. T., Bioconjugate Techniques, Academic Press, San Diego, Calif. (1996)). Griffiths et al., WO 96/40245 (published Dec. 19, 1996) and Hermanson, G. T., Bioconjugate Techniques, Academic Press, San Diego, Calif. (1996), both of which are entirely incorporated herein by reference, provide several methods for conjugating binding and capture moieties and for conjugating complementary binding partners and therapeutic agents.

Bioconjugates can be characterized and assayed for the properties of their individual components in vitro or in vivo. For example, in a particular embodiment, bioconjugates can be assayed for binding of the binding moiety to the intended suspended formed element of the blood and therapeutic activity of the therapeutic agent. In another embodiment, bioconjugates can be assayed for binding of the binding moiety to the intended suspended formed element of the blood and binding of the capture moiety to the intended complementary binding partner. In yet another embodiment, bioconjugates can be assayed for binding of the complementary binding partner to the intended capture moiety and therapeutic activity of the therapeutic agent.

In one embodiment, bioconjugates can also be assayed for sustained delivery to the circulation of a patient by evaluating the pharmacodynamics of the bioconjugate in appropriate animal models. A prolonged pharmacodynamic pattern for therapeutic agent in its conjugated state in comparison to its unconjugated state can be a measure of sustained delivery. For example, a radiolabelled form of an agent that is usually rapidly cleared from circulation (e.g., heparin, hirudin), either unconjugated or conjugated to a binding moiety (e.g., chimeric 7E3 Fab), can be injected into an animal. A significant prolongation of lifetime in circulation (confirmed as suspended formed element-bound (e.g., platelet-bound) using suitable techniques) would establish sustained delivery to the circulation. Bioconjugates useful in the present invention are those that can be used for sustained delivery of a therapeutic agent to the circulation of a patient.

Thus, the invention also relates to novel bioconjugates and their use for sustained delivery of a therapeutic agent to the circulation of a patient.

The invention further relates to bioconjugates and their use in the manufacture of a medicament for sustained delivery to the circulation of a patient.

In a particular embodiment, a bioconjugate comprising chimeric 7E3 Fab or Fab′ and heparin is administered to a patient at a predetermined effective amount for sustained release to the circulation of the patient.

In another embodiment, a bioconjugate comprising chimeric 7E3 Fab or Fab′ and chimeric antibody cA2 (or an antigen-binding fragment thereof) is administered to a patient at a predetermined effective amount for sustained release to the circulation of the patient.

Administration

Bioconjugates can be administered to a patient in a variety of ways. The routes of administration include intradermal, transdermal (e.g., in slow release polymers), intramuscular, intraperitoneal, intravenous including infusion and/or bolus injection, subcutaneous, oral, topical, epidural, buccal, rectal, vaginal and intranasal routes. Other suitable routes of administration can also be used, for example, to achieve absorption through epithelial or mucocutaneous linings. Bioconjugates can also be administered by gene therapy, wherein a DNA molecule encoding a particular bioconjugate is administered to the patient, e.g., via a vector, which causes the particular bioconjugate to be expressed and secreted at therapeutic levels in vivo. For example, immunoconjugates useful in the present invention can be administered by gene therapy, wherein a DNA molecule encoding a particular immunoconjugate is administered to the patient, e.g., via a vector, which causes the immunoconjugate to be expressed and secreted at therapeutic levels in vivo. In addition, bioconjugates can be administered together with other components of biologically active agents, such as pharmaceutically acceptable surfactants (e.g., glycerides), excipients (e.g., lactose), carriers, diluents and vehicles. If desired, certain sweetening, flavoring and/or coloring agents can also be added.

Bioconjugates useful in the present invention can be administered prophylactically or therapeutically to an individual prior to, simultaneously with or sequentially with other therapeutic regimens or agents (e.g., multiple drug regimens), in a predetermined effective amount. Bioconjugates that are administered simultaneously with other therapeutic agents can be administered in the same or different compositions.

For parenteral (e.g., intravenous, subcutaneous, intramuscular) administration, bioconjugates can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation can be sterilized by commonly used techniques.

Suitable pharmaceutical carriers are described in Remington's Pharmaceutical Sciences, A. Osol, a standard reference text.

For example, a parenteral composition suitable for administration by injection is prepared by dissolving 1.5% by weight of active ingredient in 0.9% sodium chloride solution.

The term “predetermined effective amount”, as used herein, refers to that amount of bioconjugate which has been determined to provide a sustained therapeutically effective amount of therapeutic agent to the circulation of a patient. According to the method, the ability of a bioconjugate to provide sustained delivery is determined. Reference to a predetermined effective amount subsumes a determination of sustained delivery or selection of an effective amount which has been determined to be suitable for sustained delivery. The term “therapeutically effective amount” refers to that amount of therapeutic agent sufficient for therapeutic efficacy (e.g., an amount sufficient for significantly reducing or eliminating symptoms associated with a particular disease or disorder). Advantageously, due to sustained delivery, a therapeutically effective amount of therapeutic agent provided with a predetermined effective amount of bioconjugate can be equivalent to or less than the amount of unconjugated therapeutic agent which is administered to a patient to obtain therapeutic benefit.

The dosage administered to an individual will vary depending upon a variety of factors, including the pharmacodynamic characteristics of the particular bioconjugate, and its mode and route of administration; size, age, sex, health, body weight and diet of the recipient; nature and extent of symptoms of the disease or disorder being treated, kind of concurrent treatment, frequency of treatment, and the effect desired.

A prolonged therapeutically effective range for a therapeutic agent can be obtained by administering a predetermined effective amount of bioconjugate that is equal to the therapeutically effective amount of the unconjugated therapeutic agent. In this case, the therapeutic agent will persist in the circulation for a sustained (prolonged) period in comparison to unconjugated therapeutic agent. A similar therapeutically effective range for a therapeutic agent can be obtained by administering a predetermined effective amount of bioconjugate that is less than the therapeutically effective amount of the unconjugated therapeutic agent.

Bioconjugates can be administered in single or multiple doses depending upon factors such as nature and extent of symptoms, kind of concurrent treatment and the effect desired. Thus, other therapeutic regimens or agents can be used in conjunction with the methods and bioconjugates of the present invention. Adjustment and manipulation of established dosage ranges are well within the ability of those skilled in the art.

A second or subsequent administration is preferably during or immediately prior to relapse or a flare-up of the disease or symptoms of the disease or disorder. For example, second and subsequent administrations can be given between about one day to 30 weeks from the previous administration. Two, three, four or more total administrations can be delivered to the individual, as needed. The terms “reoccurrence”, “flare-up” or “relapse” are defined to encompass the reappearance of one or more symptoms of the disease or disorder state.

The present invention will now be illustrated by the following example, which is not intended to be limiting in any way.

### EXAMPLE

### Example

Quantitation of Platelet Bound Abciximab in Abciximab-Treated Patients

Flow cytometry was utilized throughout an abciximab trial to monitor the presence and distribution of platelet bound abciximab (chimeric 7E3 Fab). Measurements were attained at the following time points: prior to dosing, during infusion of abciximab (30 min and 12 hrs post-bolus) and after cessation of therapy (1, 3, 8 and 15 days post bolus). Platelet bound abciximab was detected using a fluorescein conjugated rabbit anti-abciximab probe that was specific for the murine variable region of abciximab.

Only a single population of platelets was observed throughout the 15 day period and the fluorescence intensity of this population gradually decreased over time indicating that abciximab was re-equilibrated onto new platelets entering the circulation. Additionally, at both 8 and 15 days after dosing, the platelets maintained a significant level of fluorescence intensity. The median value of fluorescence intensity at 8 days was 30-fold higher than the baseline value and 14-fold higher than baseline at 15 days after dosing. In order to estimate the amount of abciximab remaining on the surface of platelets in these patients, a radiometric assay was utilized and a standard curve was created by plotting fluorescence intensity against molecules of abciximab bound per platelet. By extrapolation from this standard curve, the median level of abciximab binding was estimated to be approximately 31,600 molecules per platelet at 8 days and at 15 days to be 12,700 molecules per platelet. These numbers correspond to approximately 31.6% and 12.7% saturation of the GPIIb/IIIa receptors on platelets given an average GPIIb/IIIa receptor density of 100,000.

Materials and Methods

Materials

Tris Buffered Saline (TBS) (0.05 M Tris, 0.15 M NaCl, pH 7.5) was used in the radiometric assay. The platelet wash buffer, PBS-ACD, was prepared by adding 100 mL of 10× Dulbecco's PBS and 150 mL ACD solution (22 g Trisodium citrate, 8 g citric acid, 24.5 g dextrose in 1 liter dH₂o) to 750 mL dH₂O, pH to 7.4. Bovine serum albumin (1.0 g) was then added for a final concentration of 0.1% (w/v). Glycine Quenching Solution (50 mM Tris Base, 10 mM glycine, 150 mM NaCl, pH 7.4) was utilized in the flow cytometric staining procedure. Fluorescein labeled beads (2μ and 8μ) were used to calibrate the FACSCAN flow cytometer. The 2μ beads were obtained from Polysciences Inc. (cat. #18604) and the 8μ bead from Flow Cytometry Standards Corporation (cat. #891). Apyrase Grade III was supplied by Sigma (cat. no. A-7647), PGE₁ was also obtained from Sigma (cat. no. P-5515).

Preparation of Platelet Rich Plasma (PRP)

Samples were prepared and platelet rich plasma was prepared as described in Wagner et al., Blood, 88(3): 907-914 (1996). The blood for this study was obtained in citrate and the PRP stored in polypropylene tubes. The Coulter Counter ZM was calibrated using 5μ micro spheres. Additionally, a study was performed to correlate the platelet counts which were obtained with those obtained by a controlled clinical laboratory. The platelet counts in the clinical laboratory were on average 12% higher than those obtained in the study.

Radioimmunoassay (RIA) for the Quantification of Abciximab Bound Per Platelet

A 17-point standard curve was generated to compare the number of abciximab molecules bound per platelet at varying concentrations of abciximab. The procedure was a modified abciximab receptor blockade assay using varying concentrations of ¹²⁵I-abciximab (Wagner, C. L. et al., Blood, 88: 907-914 (1996)). First, a 400 μg/mL stock solution of ¹²⁵I-abciximab was prepared by adding 400 μL of ¹²⁵I-abciximab to a tube containing 3.6 mL Tris Buffered Saline (TBS) and 1.0 mL of 2.0 mg/mL abciximab. This stock solution was then used as follows (Tables 1 and 2).

TABLE 1
Dilution of ¹²⁵I-abciximab

| ¹²⁵I-abciximab | Volume of 400 μg/mL | Volume of |
|---|---|---|
| Concentration (μg/mL) | ¹²⁵I-abciximab (μL) | TBS (μL) |
| 50.0 | 50 | 350 |
| 45.0 | 45 | 355 |
| 40.0 | 40 | 360 |
| 35.0 | 35 | 365 |
| 30.0 | 30 | 370 |
| 25.0 | 25 | 375 |
| 23.0 | 23 | 377 |
| 20.0 | 20 | 380 |
| 18.0 | 18 | 382 |
| 15.0 | 15 | 385 |
| 13.0 | 13 | 387 |
| 10.0 | 10 | 390 |

TABLE 2
Dilution of Low Concentration Solutions of ¹²⁵I-abciximab

| ¹²⁵I-abciximab | Volume of | Volume of |
|---|---|---|
| Concentration (μg/mL) | ¹²⁵1-abciximab | TBS (μL) |
| 7.5 | 60 μL of 50 μg/mL | 340 |
| 5.0 | 40 μL of 50 μg/mL | 360 |
| 2.5 | 40 μL of 25 μg/mL | 360 |
| 1.25 | 20 μL of 25 μg/mL | 380 |
| 0 | 0 | 400 |

The assay was performed by adding 40 μL of each ¹²⁵I-abciximab concentrations to 360 μL aliquots of PRP (1/10 dilution of ¹²⁵I-abciximab) in 1.5 mL polypropylene microcentrifuge tubes. After 30 min at room temperature, triplicate 100 μL aliquots of each suspension were overlaid onto 200 μL cushions of 30% sucrose (w/v). The tubes were centrifuged in the microcentrifuge at maximum speed (10,000 rpm) for 5 min. The pellets were transected from the tubes, and the pellets and supernatants counted on the gamma counter. The molecules per platelet were calculated as follows:

$$\begin{matrix}Molecules abciximab per platelet = \frac{( fraction {}^{125} I - abciximab bound ) ( final abciximab conc . [ μ g / mL ] ( 0.1 mL ) ( 1.26 \times {10}^{13} molecules \text{/} μ g )}{( platelet concentration in cells \text{/} μ L ) ( 90 μ L )} \\ fraction {}^{125} I - abciximab bound : \frac{CPM pellet}{( CPM pellet + CPM supernatant )}\end{matrix}$$

Using the GRAPHPAD PRISM program, the molecules of abciximab per platelet were graphed (y-axis) versus the concentration of abciximab (x-axis). Linear regression was performed to obtain the equation of the line.

Flow Cytometric (FC) Determination of Amount of Abciximab Bound per Platelet

The same dilutions of abciximab shown in Tables 1 and 2 were prepared using an unlabeled stock of 400 μg/mL abciximab. The abciximab dilutions were prepared at the same time as the ¹²⁵I-abciximab dilutions to reduce the level of experimental error when changing the volumes of the pipetors.

Using round bottom 1.5 mL cryovials (polypropylene) 360 μL aliquots of PRP were incubated with 40 μL each of the above abciximab dilutions. After 30 min at room temperature, 10 nM PGE₁ and 0.1 U/mL apyrase was added to prevent platelet activation during centrifugation. The PRP was then centrifuged at 500×g for five minutes at room temperature. The supernatant was aspirated and the pellet resuspended in PBS-ACD containing 10 nM PGE₁ and 0.1 U/mL apyrase. The platelet suspension was repelleted, then the supernatant discarded and the platelet resuspended in autologous plasma spiked with 10 nM PGE₁ and 0.1 U/mL apyrase.

To detect platelet bound abciximab, 40 μg/mL FITC-rabbit anti-abciximab was added to a 50 μL aliquot of the treated PRP samples in amber 1.5 mL microcentrifuge tubes. After 5 min at room temperature, the cells were then fixed with 50 μL of 2% formalin in PBS. Following another 5 min incubation at room temperature, 100 μL of glycine quenching solution was added. The samples were stored at 4° C. overnight.

Flow cytometric analysis was performed using a Becton Dickinson FACSCAN flow cytometer equipped with a 15 m Watt argon laser tuned to a frequency of 488 nm. Fluorescein emission was measured through a bandpass filter 530 nM with a 30 nM bandwidth. A total of 5,000 events were collected for each sample and the platelet population was selected based on forward versus side scatter profiles. The geometric median fluorescence for each sample was determined and these results were plotted on the y-axis versus the concentration of abciximab (x-axis). The equation of the line was then calculated.

Correlation of the RIA and the FC Assays

GRAPHPAD PRISM program provides numerical values (x and y) for the calculated lines. In both assays, as graphed, the x-values corresponded to the concentration of abciximab added. For each abciximab concentration, the corresponding y-values obtained from the RIA (molecules/platelet) were plotted against the y-values obtained from the FC assay (median fluorescence channel number). The data was then graphed with median fluorescence channel number on the x-axis and molecules abciximab/platelet on the y-axis. Linear regression was calculated based on this comparison. Using this equation, the molecules of abciximab per platelet was calculated from the median fluorescence channel number.

Extrapolation of Data to the Patient Data

Forty-one patients participating in a single center, randomized trial were treated with clinical grade chimeric 7E3 Fab (Centocor, Inc., Malvern, Pa.; also referred to as ReoPro®; the chimeric 7E3 Fab is also available as abciximab, Eli Lilly and Co.). Patients included normal volunteers and patients with stable coronary artery disease. Patients received oral aspirin (325 mg p.o.) at least 4 hours, but not greater than 24 hours prior to administration of chimeric 7E3 Fab. Patients received one of the following doses: (a) a 0.25 mg/kg bolus plus a 10 μg/minute infusion for 12 hours; or (b) a 0.25 mg/kg bolus plus a 0.125 μg/kg/min infusion for 12 hours. Patients greater than 80 kg received the 0.25 mg/kg bolus plus a 10 μg/minute infusion for 12 hours. Patients weighing less than 70 kg or weighing between 70-80 kg were randomized to receive either dose regimen (a) or dose regimen (b) as indicated above.

Blood samples were obtained from patients into citrate anticoagulant at several timepoints before and after dosing with abciximab (baseline, 30 minutes, 12 hrs, and at 1, 3, 8 and 15 days after bolus). Platelet rich plasma was prepared immediately and the samples were stained with FITC-labeled rabbit anti-abciximab (40 μg/mL), then fixed in 1% formalin followed by the addition of quenching solution. These samples were analyzed by flow cytometry within 48 hrs of collection and preparation. After identifying the platelet population using a dot plot of forward scatter and side scatter, a gate was placed around the single platelet population. If greater than 50% of the total events acquired fell within this gate, the sample was considered valid. Using the valid data from all 41 patients, median fluorescence intensity at 8 days (n=36) and 15 days (n=38) was calculated. These values were then plugged into the equation described above to determine the molecules abciximab/platelet.

Flow Cytometric Quality Control

The flow cytometer was calibrated using two different fluorescein labeled beads. The beads were analyzed by flow cytometry to determine the appropriate instrument gain settings and to compensate for instrument drift on a daily basis. The gain settings for side scatter and FL1 (FITC fluorescence) were adjusted as needed so that each day the peak channel number of the beads remained consistent (±5%). Once the gains were established, 5,000 events were collected for each bead control and saved on disc. The fluorescence intensity was recorded each day as well as the gain settings used to obtain these results.

Results

The distribution of abciximab on the circulating platelet population was monitored for 15 days post-abciximab bolus using fluorescence activated cell sorting (FACS). Measurements were collected at baseline, during abciximab infusion (0.5 and 12 hours post-abciximab bolus), and after abciximab treatment (1, 3, 8 and 15 days post-abciximab bolus).

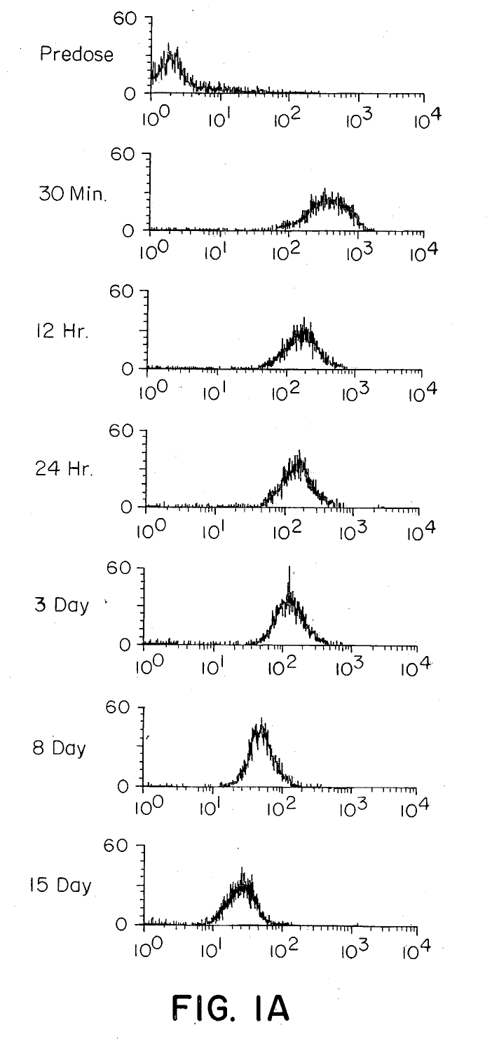

To determine the distribution of platelet-bound abciximab, citrate anticoagulated blood was collected from patients at several time points before and after administration of abciximab (0.25 mg/kg bolus plus 0.125 μg/kg/min 12 hr infusion or 0.25 mg/kg bolus plus 10 μg/min 12 hour infusion, as described above). Platelet rich plasma samples were stained with 40 μg/mL of FITC-labeled anti-abciximab and fixed with 1% formalin. The fluorescence histograms of a representative patient are illustrated at predose and at 30 minutes, 12 hours, 24 hours, 3 days, 8 days, and 15 days post-treatment (FIGS. 1A and 1B).

Platelet-bound abciximab was detected with a fluorescein-conjugated rabbit anti-abciximab reagent that interacts exclusively with the murine portion of the molecule. After staining, the platelets were formalin-fixed in order to eliminate any equilibration of abciximab occurring in vitro. For each sample, single, intact platelets were identified by the forward versus side scatter profile and gates were set around the single cell population in order to eliminate debris and platelet micro aggregates. If fewer than 50% of the events that were collected fell within this gate, the sample was deemed unacceptable and the data were not included in the statistical analysis.

The fluorescence histograms of platelet samples from two representative patients are diagrammed in FIG. 1A (patient 01017) and FIG. 1B. The fluorescence histogram of the platelets attained at baseline illustrate low endogenous fluorescence intensity prior to abciximab treatment. However, the fluorescent histograms at 30 minutes post-abciximab bolus displayed a unimodal pattern of highly fluorescent platelets, confirming that abciximab was uniformly bound to the entire platelet population. FACS analysis at time points when there was no free abciximab in the circulation (24 hour, 3, 8 and 15 days post-abciximab bolus) all exhibited a unimodal cell population that gradually diminished in relative fluorescent intensity, indicative that the level of abciximab molecules per platelet gradually decreased over time. It is also important to note that the platelet population remained unimodal throughout the 15 day monitoring period (i.e., no separate population of non-abciximab coated platelets were detected), demonstrating that abciximab was continuously re-equilibrating among old and new platelets entering the circulation. The persistence of a single fluorescent population throughout the 15 day period and the progressive reduction in the level of fluorescence intensity over time provide strong evidence that abciximab does equilibrate onto new platelets entering the circulation. Conversely, if abciximab did not dissociate from the GPIIb/IIIa receptors, a negative abciximab-staining platelet peak would appear, and the fluorescence intensity of the abciximab staining peak would not decrease, since all GPIIb/IIIa receptors on these platelets would be occupied with abciximab. However, the number of cells within this population would decrease as they are cleared from the system. Other evidence that supports the re-equilibration of abciximab onto new platelets entering the circulation is that abciximab is detected on circulating platelets beyond the normal platelet lifespan of 7 to 10 days.

To detect platelet bound abciximab in patients at various times after dosing, a flow cytometric analysis of platelets was performed. FITC-conjugated anti-abciximab (40 μg/mL) was added to platelet rich plasma samples to detect platelet bound abciximab in patients at various times after dosing. The median fluorescence channel numbers obtained by flow cytometry were graphed to illustrate the variability between patients.

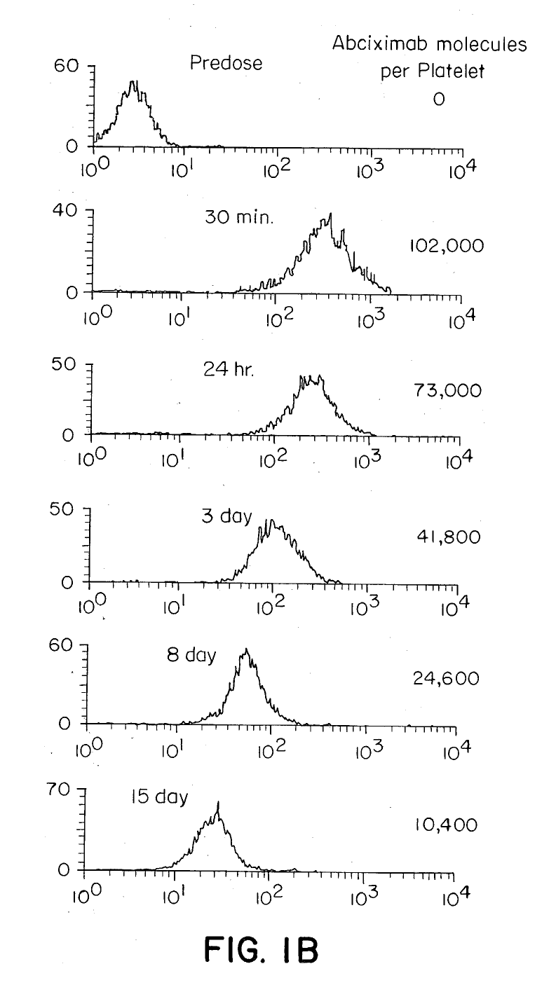

The persistence of abciximab on platelets at 8 and 15 days after dosing was observed on almost all of the patients in the trial. The fluorescence values obtained from each patient at these time periods are illustrated in FIG. 2. The median result of all valid patient data is displayed to the right of the populations. A majority of the patients (32 out of 36 with valid samples) had fluorescent values ranging from 40 to 100 at 8 days after dosing. These values are significantly higher than the fluorescence value of 2 obtained at baseline. Similarly, 33 out of 38 patients with valid samples at 15 days displayed detectable levels of fluorescence. The median level of fluorescence (27.63) at 15 days was approximately 14-fold higher than the baseline level.

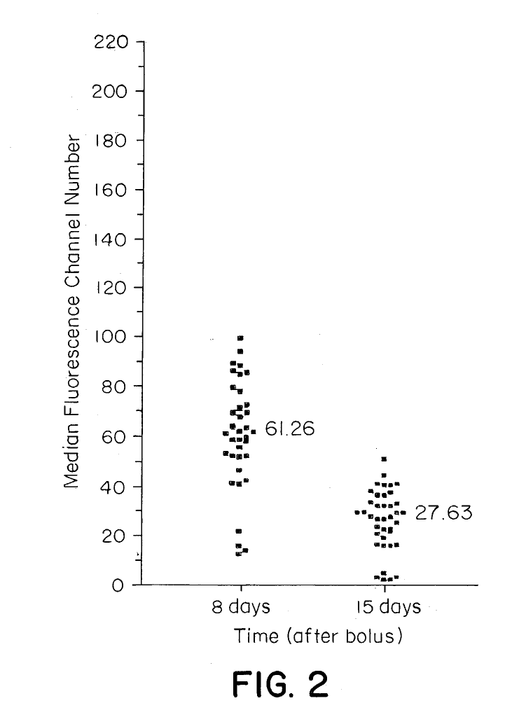

To determine the amount of abciximab that corresponds to these fluorescence values, the lot of probe used in the study was calibrated using a radiometric assay. A binding isotherm of ¹²⁵I-abciximab was generated on platelets in PRP from a normal human donor. The results from a representative assay are shown in FIG. 3A. The higher abciximab concentrations formed a sigmoidal plat with saturation occurring at approximately 2.5-3.0 μg/mL abciximab. In order to use this data to quantify the amount of abciximab bound/platelet, only the linear region was used. The 12 point curve included abciximab concentrations ranging from 0 to 2.5 μg/mL. Using GRAPHPAD PRISM program, linear regression of the data was performed and x, y coordinates for the line was also extrapolated by the program. The data were very linear at this concentration range with an r² value of 0.999.

In the radiometric assay (FIG. 3A), varying concentrations of radiolabeled abciximab were added to platelets at varying concentrations. After 30 minutes, the unbound fraction was removed by centrifugation through a sucrose cushion. The average number of abciximab molecules bound per platelet was calculated and plotted against the original concentration of abciximab in the sample. Linear regression was performed to obtain the equation of the line.

In the flow cytometric assay (FIG. 3B), the platelets were treated with varying levels of abciximab. The platelets were washed twice and resuspended in plasma. FITC-labeled anti-abciximab (40 μg/mL) was added and after 5 min the cells were fixed with 1% formalin. The platelets were analyzed by flow cytometry and the median fluorescence intensity determined for each sample. The fluorescence was plotted against the concentration of added abciximab and the equation of the line calculated.

For each abciximab concentration, the corresponding y-values obtained from the radiometric assay (molecules/platelet) were plotted against the y-values obtained from the flow cytometric assay (median fluorescence channel number). Linear regression was calculated based on this comparison. Using this equation, the molecules of abciximab per platelet could be calculated from the median fluorescence channel number.

To assure that the level of fluorescence intensity for a given amount of platelet bound abciximab remained constant throughout these analyses, two bead standards were analyzed. Throughout the analysis of patient samples, the instrument was calibrated using 2μ and 8μ micro spheres conjugated with fluorescein. The instrument gains were adjusted daily to assure that the fluorescence intensity of the beads remained consistent throughout the study. These same beads were also used on the day that the probe was calibrated. The bead results are presented in Table 3.

TABLE 3
Bead Control Data

| | Fluorescence 1 (FITC) | | Side Scatter (SCC) | |
|---|---|---|---|---|
| | 2μ beads | 8μ beads | 2μ beads | 8μ beads |
| Mean | 3630.27 | 707.06 | 92.32 | 418.94 |
| SD | 37.38 | 36.29 | 1.84 | 14.15 |
| % CV | 1.05% | 5.13% | 2.00% | 3.38% |
| Range | 3554.31 | 634.59 | 88.64 | 390.64 |
| (±2 SD) | 3706.23 | 779.74 | 96.01 | 447.23 |
| Date of | 3555 | 736.53 | 88.17 | 406.79 |
| Calibration | | | | |

Over the period of sample analysis, the % CV of the 2μ beads was 1.05% and the 8μ beads was 5.13%. The fluorescence intensity of the beads on the day of the probe calibration fell within the vary narrow range of 2 standard deviations. These results indicate that the patient data obtained on different days can be accurately extrapolated on the in vitro calibration curves.

A simultaneous flow cytometric assay was performed using the same concentrations of abciximab (unlabeled) that were used in the radiometric assay. The excess abciximab was washed off the platelets and the membrane bound abciximab was detected using the same lot of fluoresceinated probe that was used for patient samples. The results obtained from this assay are illustrated in FIG. 3B. As with the radiometric assay, saturation of the fluorescence appeared to occur at approximately 2.5-3.0 μg/mL abciximab (data not shown). Therefore, only the linear portion of the data was used. The linear regression and x, y coordinates were calculated using Graphpad Prism.

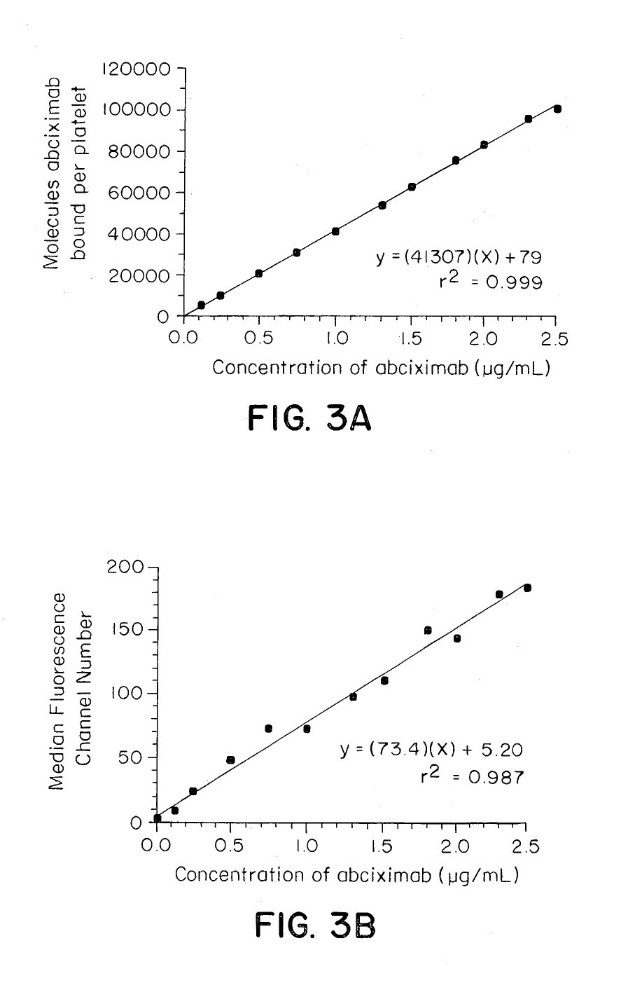

In order to correlate the molecules per platelet with the observed level of fluorescence intensity, the two assays were graphed against each other. In each assay the x-values represented the concentration of abciximab in μg/mL. Because these x-values were identical, the corresponding y-values were plotted against each other. FIG. 4 illustrates the final linear regression analysis. The equation resulting from this analysis was y=(563)(x)−2848 where y is the molecules of abciximab/platelet and x is the median fluorescence channel number.

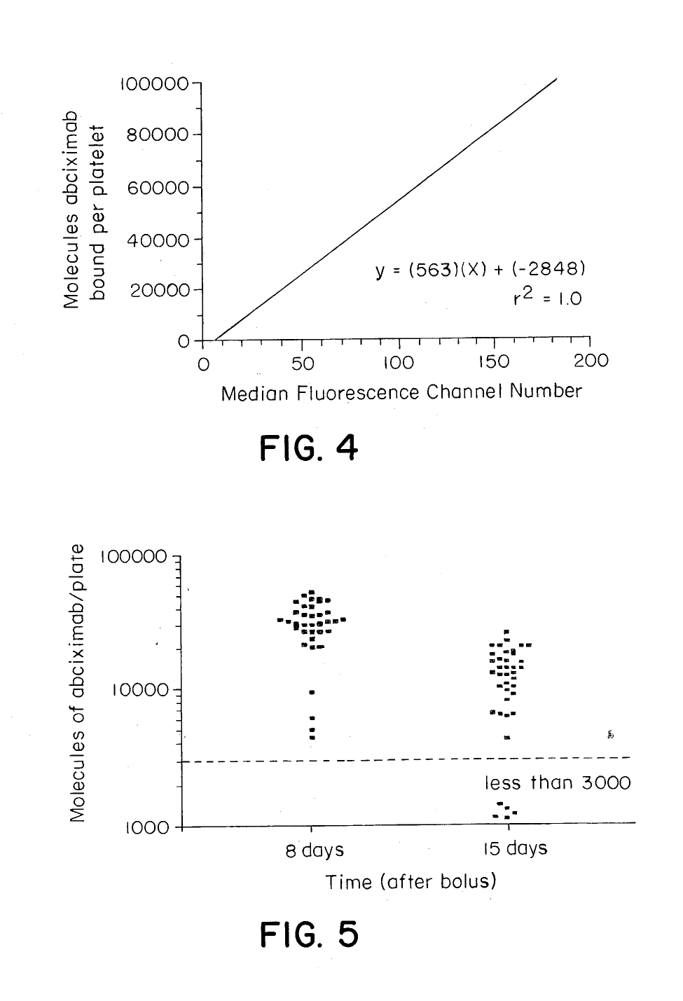

The molecules of abciximab bound per platelet was calculated for each individual patient at 8 days and 15 days. Using the results obtained from 36 patients, at 8 days, the median density of abciximab was 31,600. The actual patient values range from 4,000 to 52,000 molecules per platelet. The data from 38 patients revealed that, at 15 days there were approximately 12,700 molecules bound per platelet. This covers a range of 0 to 26,000 molecules/platelet. The data from each individual patient are shown in FIG. 5. The median fluorescence and median density are shown in Table 4.

TABLE 4

| | Number of | Median | Molecules/ |
|---|---|---|---|
| Time | Patients | fluorescence | Platelet |
| 8 d | 36 | 61.26 | 31,600 |
| 15 d | 37 | 27.63 | 12,700 |

Conclusion

Using flow cytometry, measurable amounts of abciximab remaining on the platelets 15 days after abciximab administration have been detected. This calibration assay allows one to quantitate the amount of abciximab remaining on the platelet surface for up to two weeks after dosing. Patients enrolled in this abciximab study, had approximately 31,600 molecules of abciximab remaining on the platelets at 8 days and 12,700 at 15 days.

The average circulating lifetime of a platelet is 7 to 9 days. Therefore, at 15 days after abciximab administration, the originally-circulating platelets would have been replaced by new platelets entering circulation. The persistence of platelet-bound abciximab at prolonged times provide strong evidence that abciximab continuously redistributes among circulating platelets including those newly entered into circulation. A corollary to this pharmacodynamic profile is that platelets have equivalent numbers of bound abciximab throughout the prolonged recovery period. In addition, the gradual recovery from receptor blockade (gradually diminishing receptor blockade) is a property of all of the platelets in circulation and is not due to an averaging effect of new platelets that have entered circulation after cessation of abciximab administration.

Equivalents

Those skilled in the art will be able to recognize, or be able to ascertain, using no more than routine experimentation, many equivalents to the specific embodiments of the invention described herein. Such equivalents are intended to be encompassed by the following claims.

What is claimed is:

1. A method for sustained delivery of a therapeutic agent to the circulation of a patient comprising administering to the patient a predetermined effective amount of a bioconjugate, the bioconjugate comprising a binding moiety which selectively binds to a suspended formed element of the blood and the therapeutic agent, wherein the binding moiety is a chimeric 7E3 antibody or an antigen-binding fragment thereof and the therapeutic agent is not a thrombolytic agent.

2. The method of claim 1 wherein the therapeutic agent is an antibody or an antigen-binding fragment thereof.

3. The method of claim 1 wherein the therapeutic agent is heparin.

4. A method for sustained delivery of a therapeutic agent to the circulation of a patient comprising administering to the patient a predetermined effective amount of a bioconjugate, the bioconjugate comprising a binding moiety which selectively binds to a suspended formed element of the blood and the therapeutic agent, wherein the binding moiety is a chimeric 7E3 Fab or Fab′ fragment and the therapeutic agent is not a thrombolytic agent.

5. The method of claim 4 wherein the therapeutic agent is an antibody or antigen-binding fragment thereof.

6. The method of claim 4 wherein the therapeutic agent is heparin.

7. A method for sustained delivery of a therapeutic agent to the circulation of a patient comprising administering to the patient a predetermined effective amount of a bioconjugate, the bioconjugate comprising a binding moiety which selectively binds to a suspended formed element of the blood and the therapeutic agent, wherein the binding moiety is an antibody or antigen-binding fragment thereof having specificity for glycoprotein IIb/IIIa, said antibody or fragment having the epitopic specificity of murine monoclonal antibody 7E3, and the therapeutic agent is not a thrombolytic agent.

8. The method of claim 7 wherein the therapeutic agent is an antibody or antigen-binding fragment thereof.

9. The method of claim 7 wherein the therapeutic agent is heparin.

10. A method for sustained delivery of a therapeutic agent to the circulation of a patient comprising the steps of:
a) administering to the patient a predetermined effective amount of a first bioconjugate comprising a binding moiety which selectively binds to a suspended formed element of the blood and a capture moiety, wherein said binding moiety is a chimeric 7E3 antibody or an antigen-binding fragment thereof and said capture moiety comprises a binding site for a complementary binding partner; and
b) administering to the patient a predetermined effective amount of a second bioconjugate comprising a complementary binding partner and a therapeutic agent,
whereby sustained delivery of said therapeutic agent to the circulation of said patient occurs.

11. The method of claim 10 wherein the therapeutic agent is an antibody or antigen-binding fragment thereof.

12. The method of claim 10 wherein the therapeutic agent is heparin.

13. The method of claim 10 wherein the capture moiety is an avidin molecule and the complementary binding partner is a biotin molecule.

14. A method for sustained delivery of a therapeutic agent to the circulation of a patient comprising the steps of:
a) administering to the patient a predetermined effective amount of a first bioconjugate comprising a binding moiety which selectively binds to a suspended formed element of the blood and a capture moiety, wherein said binding moiety is a chimeric 7E3 Fab or Fab′ fragment and said capture moiety comprises a binding site for a complementary binding partner; and
b) administering to the patient a predetermined effective amount of a second bioconjugate comprising a complementary binding partner and a therapeutic agent,
whereby sustained delivery of said therapeutic agent to the circulation of said patient occurs.

15. The method of claim 14 wherein the therapeutic agent is an antibody or antigen-binding fragment thereof.

16. The method of claim 14 wherein the therapeutic agent is heparin.

17. The method of claim 14 wherein the capture moiety is an avidin molecule and the complementary binding partner is a biotin molecule.

18. A method for sustained delivery of a therapeutic agent to the circulation of a patient comprising the steps of:
a) administering to the patient a predetermined effective amount of a first bioconjugate comprising a binding moiety which selectively binds to a suspended formed element of the blood and a capture moiety, wherein said binding moiety is an antibody or antigen-binding fragment thereof having specificity for glycoprotein IIb/IIIa, said antibody or fragment having the epitopic specificity of murine monoclonal antibody 7E3, and said capture moiety comprises a binding site for a complementary binding partner; and
b) administering to the patient a predetermined effective amount of a second bioconjugate comprising a complementary binding partner and a therapeutic agent,
whereby sustained delivery of said therapeutic agent to the circulation of said patient occurs.

19. The method of claim 18 wherein the therapeutic agent is an antibody or antigen-binding fragment thereof.

20. The method of claim 18 wherein the therapeutic agent is heparin.

21. The method of claim 18 wherein the capture moiety is an avidin molecule and the complementary binding partner is a biotin molecule.

22. A method of preparing a bioconjugate for sustained delivery of a therapeutic agent to the circulation of a patient comprising the steps of:
a) conjugating a binding moiety which selectively binds to a suspended formed element of the blood to the therapeutic agent, wherein said binding moiety is a chimeric 7E3 antibody or an antigen-binding fragment thereof, thereby producing a bioconjugate; and
b) screening said bioconjugate for sustained delivery of the therapeutic agent,
whereby a bioconjugate for sustained delivery of said therapeutic agent to the circulation of a patient is produced.

23. A method of preparing a bioconjugate for sustained delivery of a therapeutic agent to the circulation of a patient comprising the steps of:
a) conjugating a binding moiety which selectively binds to a suspended formed element of the blood to the therapeutic agent, wherein said binding moiety is a chimeric 7E3 Fab or Fab′ fragment, thereby producing a bioconjugate; and
b) screening said bioconjugate for sustained delivery of the therapeutic agent,
whereby a bioconjugate for sustained delivery of said therapeutic agent to the circulation of a patient is produced.

24. A method of preparing a bioconjugate for sustained delivery of a therapeutic agent to the circulation of a patient comprising the steps of:
a) conjugating a binding moiety which selectively binds to a suspended element of the blood to the therapeutic agent, wherein said binding moiety is an antibody or antigen-binding fragment thereof having specificity for glycoprotein IIb/IIIa, said antibody or fragment having the epitopic specificity of murine monoclonal antibody 7E3, thereby producing a bioconjugate; and
b) screening said bioconjugate for sustained delivery of the therapeutic agent,
whereby a bioconjugate for sustained delivery of said therapeutic agent to the circulation of a patient is produced.

25. A bioconjugate suitable for sustained delivery of a therapeutic agent to the circulation of a patient comprising a binding moiety which selectively binds to a suspended formed element of the blood and the therapeutic agent, wherein the binding moiety is a chimeric 7E3 antibody or an antigen-binding fragment thereof and the therapeutic agent is not a thrombolytic agent.

26. A bioconjugate suitable for sustained delivery of a therapeutic agent to the circulation of a patient comprising a binding moiety which selectively binds to a suspended formed element of the blood and the therapeutic agent, wherein the binding moiety is a chimeric 7E3 Fab or Fab′ fragment and the therapeutic agent is not a thrombolytic agent.

27. A bioconjugate suitable for sustained delivery of a therapeutic agent to the circulation of a patient comprising a binding moiety which selectively binds to a suspended formed element of the blood and the therapeutic agent, wherein the binding moiety is an antibody or antigen-binding fragment thereof having specificity for glycoprotein IIb/IIIa, said antibody or fragment having the epitopic specificity of murine monoclonal antibody 7E3 and the therapeutic agent is not a thrombolytic agent.

28. A bioconjugate pair suitable for sustained delivery of a therapeutic agent to the circulation of a patient comprising:
a) a first bioconjugate comprising a binding moiety which selectively binds to a suspended formed element of the blood and a capture moiety, wherein said binding moiety is a chimeric 7E3 antibody or an antigen-binding fragment thereof and said capture moiety comprises a binding site for a complementary binding partner; and
b) a second bioconjugate comprising the complementary binding partner and the therapeutic agent.

29. A bioconjugate pair suitable for sustained delivery of a therapeutic agent to the circulation of a patient comprising:
a) a first bioconjugate comprising a binding moiety which selectively binds to a suspended formed element of the blood and a capture moiety, wherein said binding moiety is a chimeric 7E3 Fab or Fab′ fragment and said capture moiety comprises a binding site for a complementary binding partner; and
b) a second bioconjugate comprising the complementary binding partner and the therapeutic agent.

30. A bioconjugate pair suitable for sustained delivery of a therapeutic agent to the circulation of a patient comprising:
a) a first bioconjugate comprising a binding moiety which selectively binds to a suspended formed element of the blood and a capture moiety, wherein said binding moiety is an antibody or antigen-binding fragment thereof having specificity for glycoprotein IIb/IIIa, said antibody or fragment having the epitopic specificity of murine monoclonal antibody 7E3 and said capture moiety comprises a binding site for a complementary binding partner; and
b) a second bioconjugate comprising the complementary binding partner and the therapeutic agent.

31. A method of preparing a bioconjugate pair for sustained delivery of a therapeutic agent to the circulation of a patient comprising the steps of:
a) conjugating a binding moiety which selectively binds to a suspended formed element of the blood to a capture moiety, wherein said binding moiety is a chimeric 7E3 antibody or an antigen-binding fragment thereof and said capture moiety comprises a binding site for a complementary binding partner, thereby producing a first bioconjugate;
b) conjugating the complementary binding partner to the therapeutic agent, thereby producing a second bioconjugate; and
c) screening said bioconjugate pair for sustained delivery of the therapeutic agent,
whereby a bioconjugate pair for sustained delivery of said therapeutic agent to the circulation of a patient is produced.

32. A method of preparing a bioconjugate pair for sustained delivery of a therapeutic agent to the circulation of a patient comprising the steps of:
a) conjugating a binding moiety which selectively binds to a suspended formed element of the blood to a capture moiety, wherein said binding moiety is a chimeric 7E3 Fab or Fab′ fragment and said capture moiety comprises a binding site for a complementary binding partner, thereby producing a first bioconjugate;
b) conjugating the complementary binding partner to the therapeutic agent, thereby producing a second bioconjugate; and
c) screening said bioconjugate pair for sustained delivery of the therapeutic agent,
whereby a bioconjugate pair for sustained delivery of said therapeutic agent to the circulation of a patient is produced.

33. A method of preparing a bioconjugate pair for sustained delivery of a therapeutic agent to the circulation of a patient comprising the steps of:
a) conjugating a binding moiety which selectively binds to a suspended formed element of the blood to a capture moiety, wherein said binding moiety is an antibody or antigen-binding fragment thereof having specificity for glycoprotein IIb/IIIa, said antibody or fragment having the epitopic specificity of murine monoclonal antibody 7E3 and said capture moiety comprises a binding site for a complementary binding partner, thereby producing a first bioconjugate;
b) conjugating the complementary binding partner to the therapeutic agent, thereby producing a second bioconjugate; and
c) screening said bioconjugate pair for sustained delivery of the therapeutic agent,
whereby a bioconjugate pair for sustained delivery of said therapeutic agent to the circulation of a patient is produced.
